(19) United States
(12) Patent Application Publication
(10) Pub. No.: US 2007/0281002 A1
(43) Pub. Date: Dec. 6, 2007

(54) LOW IRRITATION ANTIMICROBIAL CLEANING SUBSTRATE

(76) Inventors: Sara Morales, Pittsburg, CA (US); Julio Cardozo, Dublin, CA (US); Daniela Fritter, Dublin, CA (US); Michael Robbins, Marietta, GA (US)

Correspondence Address: THE CLOROX COMPANY, P.O. BOX 24305, OAKLAND, CA, 94623-1305, US

(21) Appl. No.: 11/421,277

(22) Filed: May 31, 2006

(51) Int. Cl. A61K 9/70

(52) U.S. Cl. 424/443

(57) ABSTRACT

The transfer of antimicrobial agent from a cleaning substrate to the skin can be reduced resulting in reduced skin irritancy when the viscosity of the cleaning composition containing the antimicrobial is increased in viscosity. The transfer is measured by the Simulated Hand Transfer Method. The cleaning substrate is used to clean hard surfaces, especially bathroom surfaces such as toilets, and is attached to a cleaning implement. Also provided is a method of promoting a product having reduced skin irritancy.

### BACKGROUND OF THE INVENTION

1. Field of the Invention

The present invention relates generally to a method of reducing skin irritation in cleaning substrates used for cleaning hard surfaces, such as a toilet or other bathroom surface. The invention also relates to a method of promoting the use of a cleaning substrate with low skin irritation.

2. Description of the Related Art

Numerous types of cleaning compositions, as well as holders for disposable cleaning pads, are known in the art. Illustrative are the compositions and apparatus disclosed in U.S. Pat. No. 4,852,201, U.S. Pat. No. 4,523,347, U.S. Pat. No. 4,031,673, U.S. Pat. No. 3,413,673 and U.S. Pat. No. 3,383,158. U.S. Pat. No. 6,586,385 to Wisniewski et al. discloses a concentrated cleaning formulation on a diswashing wipe. U.S. Pat. App. 2003/0100465 to Kilkenny et al. discloses antimicrobial compositions for use on wipes. U.S. Pat. No. 6,514,923 to Cheung et al. and U.S. Pat. App. 2004/0029767 to Lichtenberg et al. disclose dilutable disinfecting cleaning compositions. U.S. Pat. No. 6,730,654 to Godfroid et al. discloses antimicrobial compositions for hard surfaces containing quaternary ammonium compounds and biguamides. U.S. Pat. App. 2001/0044395 to Aszman et al., U.S. Pat. No. 6,248,705 to Cardola et al., and U.S. Pat. No. 6,239,092 to Papasso et al. disclose quarternary ammonium compound based manual toilet bowl cleaners. U.S. Pat. No. 4,852,201 to Wundrock et al. discloses a toilet bowl cleaner having a handle with a removable cleaning pad disposed on one end. The toilet bowl cleaner also includes a cleaning solution that is contained in the pad.

When cleaner impregnated cleaning substrates are designed to be used by hand, for example in body cleansing or dishwashing, they generally contain mild ingredients to limit potential skin irritation. When cleaner impregnated cleaning substrates are attached to cleaning implements for the purpose or tough cleaning tasks, such as cleaning toilets or cleaning showers, they can contain harsh cleaning ingredients because generally skin contact is limited. When the cleaning substrates used with cleaning implements contain antimicrobial agents for the purpose of sanitizing or disinfecting hard surfaces, the substates generally have limited skin contact, however, the concentrated antimicrobial agent along with the other ingredients can cause a higher level of skin irritation even with limited skin contact.

Methods to determine skin irritation of chemicals are described for example in U.S. Pat. App. 2006/0057558 to Scott, where a solution of the chemical is applied to cultured skin. A method of reducing skin irritation using a hydrophobically modified polymer that binds surfactants is described in U.S. Pat. App. 2005/00752556 to Librizzi et al. This reference also describes measuring skin irritation using a diluted product in a trans-epithelial permeability test. A method of reducing the skin's response to irritants by adding cations that interact with the dermal layer is described in U.S. Pat. No. 6,455,076 to Hahn et al., which applies a solution to the skin of subjects. A method of reducing the skin irritation of antimicrobials in facial tissues by placing a layer of irritation-inhibiting agents over the top of the antimicrobials is described in U.S. Pat. App. 2005/0271710 to Argo et al. The problems associated with skin irritation and antimicrobial agents in cleaning substrates of the prior art can be avoiding by limiting the transfer of antimicrobial agent to the hand on incidental hand contact. It is therefore an object of the present invention to provide an antimicrobial cleaning composition in a cleaning substrate that overcomes the disadvantages and shortcomings associated with prior art examples.

### SUMMARY OF THE INVENTION

In accordance with the above objects and those that will be mentioned and will become apparent below, one aspect of the present invention comprises a method of promoting a first product comprising;
a. using a statement comparing or directing a user to compare the irritation of the first product to a second product; and
b. using a description relating to the irritation of the first product;
c. wherein the first product comprises a cleaning substrate comprising an antimicrobial agent;
d. wherein the potential incidental hand transfer of the antimirobial agent from the substrate by the Simulated Hand Transfer Method is less for the first product than for the second product; and
e. wherein the first product is used to clean hard surfaces.

In accordance with the above objects and those that will be mentioned and will become apparent below, another aspect of the present invention comprises a method of reducing the skin irritation of a disposable substrate for use on a toilet cleaning tool comprising:
a. impregnating a cleaning composition in the substrate;
b. wherein the cleaning composition comprises greater than 5% antimicrobial agent;
c. wherein the cleaning composition has a viscosity greater than 100 cP; and
d. wherein the potential incidental hand transfer by the Simulated Hand Transfer Method is less than 50 mg of the antimicrobial agent.

In accordance with the above objects and those that will be mentioned and will become apparent below, another aspect of the present invention comprises a method of reducing the skin irritation of a disposable substrate for use on a cleaning implement comprising:
a. limiting the potential incidental hand transfer of an antimicrobial agent from the substrate by the Simulated Hand Transfer Method to less than 50 mg of the antimicrobial agent;
b. wherein the substrate is impregnated with a cleaning composition;
c. wherein the cleaning composition comprises the antimicrobial agent; and
d. wherein the cleaning composition has a viscosity greater than 100 cP.

Further features and advantages of the present invention will become apparent to those of ordinary skill in the art in view of the detailed description of preferred embodiments below, when considered together with the attached claims.

### DETAILED DESCRIPTION OF THE INVENTION

Before describing the present invention in detail, it is to be understood that this invention is not limited to particularly exemplified systems or process parameters that may, of course, vary. It is also to be understood that the terminology used herein is for the purpose of describing particular embodiments of the invention only, and is not intended to limit the scope of the invention in any manner.

All publications, patents and patent applications cited herein, whether supra or infra, are hereby incorporated by reference in their entirety to the same extent as if each individual publication, patent or patent application was specifically and individually indicated to be incorporated by reference.

It must be noted that, as used in this specification and the appended claims, the singular forms “a,” “an” and “the” include plural referents unless the content clearly dictates otherwise. Thus, for example, reference to a “surfactant” includes two or more such surfactants.

Unless defined otherwise, all technical and scientific terms used herein have the same meaning as commonly understood by one of ordinary skill in the art to which the invention pertains. Although a number of methods and materials similar or equivalent to those described herein can be used in the practice of the present invention, the preferred materials and methods are described herein.

The cleaning substrate can be used as a disinfectant, sanitizer, and/or sterilizer. As used herein, the term “disinfect” shall mean the elimination of many or all pathogenic microorganisms on surfaces with the exception of bacterial endospores. As used herein, the term “sanitize” shall mean the reduction of contaminants in the inanimate environment to levels considered safe according to public health ordinance, or that reduces the bacterial population by significant numbers where public health requirements have not been established. An at least 99% reduction in bacterial population within a 24 hour time period is deemed “significant.” As used herein, the term “sterilize” shall mean the complete elimination or destruction of all forms of microbial life and which is authorized under the applicable regulatory laws to make legal claims as a “Sterilant” or to have sterilizing properties or qualities.

In the application, effective amounts are generally those amounts listed as the ranges or levels of ingredients in the descriptions, which follow hereto. Unless otherwise stated, amounts listed in percentage (“%'s”) are in weight percent (based on 100% active) of the cleaning composition alone, not accounting for the substrate weight. Each of the noted cleaner composition components and substrates is discussed in detail below.

As used herein, the term “substrate” is intended to include any material that is used to clean an article or a surface. Examples of cleaning substrates include, but are not limited to nonwovens, sponges, films and similar materials, which can be attached to a cleaning implement, such as a toilet cleaning device. As used herein, “disposable” is used in its ordinary sense to mean an article that is disposed or discarded after a limited number of usage events, preferably less than 25, more preferably less than about 10, and most preferably less than about 2 entire usage events.

As used herein, “wiping” refers to any shearing action that the substrate undergoes while in contact with a target surface. This includes hand or body motion, substrate-implement motion over a surface, or any perturbation of the substrate via energy sources such as ultrasound, mechanical vibration, electromagnetism, and so forth.

As used herein, the terms “nonwoven” or “nonwoven web” means a web having a structure of individual fibers or threads which are interlaid, but not in an identifiable manner as in a knitted web. Nonwoven webs have been formed from many processes, such as, for example, meltblowing processes, spunbonding processes, and bonded carded web processes.

As used herein, the term “polymer” generally includes, but is not limited to, homopolymers, copolymers, such as for example, block, graft, random and alternating copolymers, terpolymers, etc. and blends and modifications thereof. Furthermore, unless otherwise specifically limited, the term “polymer” shall include all possible geometrical configurations of the molecule. These configurations include, but are not limited to isotactic, syndiotactic and random symmetries.

The term “sponge”, as used herein, is meant to mean an elastic, porous material, including, but not limited to, compressed sponges, cellulosic sponges, reconstituted cellulosic sponges, cellulosic materials, foams from high internal phase emulsions, such as those disclosed in U.S. Pat. No. 6,525,106, polyethylene, polypropylene, polyvinyl alcohol, polyurethane, polyether, and polyester sponges, foams and nonwoven materials, and mixtures thereof.

The term “cleaning composition”, as used herein, is meant to mean and include a cleaning formulation having at least one surfactant.

The term “surfactant”, as used herein, is meant to mean and include a substance or compound that reduces surface tension when dissolved in water or water solutions, or that reduces interfacial tension between two liquids, or between a liquid and a solid. The term “surfactant” thus includes anionic, nonionic and/or amphoteric agents.

### Skin Irritation Instructions

The method of use of the inventive product may include a means for directing a user to use the inventive product. Any suitable means for directing a user to attach the cleaning substrate to the cleaning implement may be used according to the directing step of the present methods. Examples of methods of directing a user include, but are not limited to, written, visual, or verbal statements made on the product, or in stores, magazines, newspaper, radio, television, internet, and the like as advertising and/or marketing for the product. In certain embodiments, the product contains written instructions on the product directing the user to attach the cleaning substrate to the cleaning implement. Such instructions may be printed on the container, label insert, or on any additional packaging.

In certain embodiments, the methods of the present invention further comprise the step of describing to a user the reduced irritation properties associated with the product and/or use of the product. Examples of methods of describing such properties to a user include, but are not limited to, written, visual, or verbal statements made on the product, or in stores, magazines, newspaper, radio, television, internet, and the like as advertising and/or marketing for the product. In certain embodiments, the product contains written description of the reduced irritation properties associated with the product.

Any description suitable to indicate the reduced irritation associated with the product comprising the reduced skin irritation may be used according to the present invention. By way of non-limiting example, statements describing reduced irritation may include phrases such as “gentle,” “mild,” “mildness,” “reduced irritation,” “reduces irritation” “low irritation,” “lower irritation,” “less irritation,” “no irritation,” “less irritating,” “without irritating,” “non-irritating,” “reduced sting,” “reduces sting,” “low sting,” “lower sting,” “less sting,” “less stinging,” “without stinging,” “reduced stinging,” “no sting,” “non-stinging,” any of the aforementioned statements including the terms “eye,” “eyes,” “to the eyes,” “skin,” or “to the skin,” and the like, or statements comparing or directing a user to compare the irritaion of the inventive product to a second product using a description relating to the irritation of the inventive product.

In light of applicants unexpected discoveries regarding the reduced irritation associated with the inventive product, applicants have recognized the significant advantage associated with identifying a target consumer, preferably a consumer for whom a product of the present invention and the reduced irritation associated therewith would be desirable, and producing and/or promoting a product of the present invention for such target user.

Any suitable means of promoting a product for a target consumer may be used. In certain embodiments, the promoting step comprises a description of the target user on the product and/or via written, oral, visual, or other means in advertising, marketing, and the like of the product. In certain embodiments, the promoting step further comprises describing the reduced irritation associated with the product to the user.

### Cleaning Implement

In an embodiment of the invention, the cleaning implement comprises the tool assembly disclosed in Co-pending Application U.S. 2005/0066465, entitled “Cleaning Tool with Gripping Assembly for a Disposable Scrubbing Head”, filed Sep. 30, 2003. In another embodiment of the invention, the cleaning implement comprises the tool assembly disclosed in Co-pending Application U.S. 2004/0255418, entitled “Cleaning Tool with Gripping Assembly for a Disposable Scrubbing Head”, filed Jun. 23, 2003. In another embodiment of the invention, the cleaning implement comprises the tool assembly disclosed in Co-pending Application U.S. 2005/0155630, entitled “Multilayer Cleaning Pad”, filed Apr. 30, 2004.

In another embodiment of the invention, the cleaning implement comprises an elongated shaft having a handle portion on one end thereof. The tool assembly may further include a gripping mechanism that is mounted to the shaft to engage the removable cleaning substrate. Examples of suitable cleaning implements are found in U.S. Pat. App. 2003/0070246 to Cavalheiro; U.S. Pat. No. 4,455,705 to Graham; U.S. Pat. No. 5,003,659 to Paepke; U.S. Pat. No. 6,485,212 to Bomgaars et al.; U.S. Pat. No. 6,290,781 to Brouillet, Jr.; U.S. Pat. No. 5,862,565 to Lundstedt; U.S. Pat. No. 5,419,015 to Garcia; U.S. Pat. No. 5,140,717 to Castagliola; U.S. Pat. No. 6,611,986 to Seals; U.S. Pat. App. 2002/0007527 to Hart; and U.S. Pat. No. 6,094,771 to Egolf et al.

The cleaning implement holding the removable cleaning substrate may have a cleaning head with an attachment means or the attachment means may be an integral part of the handle of the cleaning implement or may be removably attached to the end of the handle. The cleaning substrate may be attached by a friction fit means, by a clamping means, by a threaded screw means, by hook and loop attachment or by any other suitable attachment means. Suitable attachment structures are described in U.S. Pat. No. 6,814,519 to Pollicicchio et al., PCT App. WO2002/071915 to Truong et al., U.S. Pat. No. 6,611,986 to Seals, PCT App. WO2001/15587 to Trenz et al., and U.S. Pat. App. 2002/0083542 to Hart. The cleaning substrate may have a rigid or flexible plastic or metal fitment for attachment to the cleaning implement or the cleaning pad may be directly attached to the cleaning implement.

### Cleaning Substrate

A wide variety of materials can be used as the cleaning substrate. The substrate should have sufficient wet strength, abrasivity, loft and porosity. Examples of suitable substrates include, nonwoven substrates, wovens substrates, hydro-entangled substrates, foams and sponges. Any of these substrates may be water-insoluble, water-dispersible, or water-soluble. Suitable substrates are described in Co-pending Application U.S. 2005/0155631, which was filed Jun. 29, 2004, entitled “Cleaning Pad with Functional Properties”, and incorporated herein.

The cleaning substrate may show minimal migration of the cleaning composition during storage. The cleaning substrate may comprise 100% thermoplastic fibers or 100% of the same thermoplastic fiber type in order to allow the more convenient bonding of layers. The cleaning substrate may also comprise some non-thermoplastic fibers, such as cellulosic fibers. The cleaning substrate may allow the cleaning composition to be used up after use on one to two tasks, for example one to two showers or toilets.

### Cleaning Composition

In one embodiment, the cleaning substrate is impregnated with a cleaning composition and is ‘wet-to-the-touch’. In another embodiment, the cleaning substrate is impregnated with a cleaning composition that is ‘dry-to-the-touch’. By ‘dry-to-the-touch’, it is meant that the substrate is free of water or other solvents in an amount that would make them feel damp or wet-to-the-touch as compared to the touch of a wet substrate, for example a wet cleaning wipe.

### Antimicrobial Agent

A wide range of quaternary compounds can be used as antimicrobial actives. Non-limiting examples of useful quaternary compounds include: (1) benzalkonium chlorides and/or substituted benzalkonium chlorides such as commercially available Barquat® (available from Lonza), Maquat® (available from Mason), Variquat® (available from Witco/Sherex), and Hyamine® (available from Lonza); (2) di(C6-C14)alkyl di short chain (C1-4 alkyl and/or hydroxyalkl) quaternary such as Bardac® products of Lonza, (3) N-(3-chloroallyl)hexaminium chlorides such as Dowicide® and Dowicil® available from Dow; (4) benzethonium chloride such as Hyamine® from Rohm & Haas; (5) methylbenzethonium chloride represented by Hyamine® 10× supplied by Rohm & Haas, (6) cetylpyridinium chloride such as Cepacol chloride available from of Merrell Labs. Examples of the suitable dialkyl quaternary compounds are di(C8-C12)dialkyl dimethyl ammonium chloride, such as didecyldimethylammonium chloride (Bardac 22), and dioctyldimethylammonium chloride (Bardac 2050). The quaternary compounds useful as cationic antimicrobial actives herein can be selected from the group consisting of dialkyldimethylammonium chlorides, alkyldimethylbenzylammonium chlorides, dialkylmethylbenzylammonium chlorides, and mixtures thereof. Other suitable cationic antimicrobial actives useful herein include diisobutylphenoxyethoxyethyl dimethylbenzylammonium chloride (commercially available under the trade name Hyamine® 1622 from Rohm & Haas) and (methyl) diisobutylphenoxyethoxyethyl dimethylbenzylammonium chloride (i.e. methylbenzethonium chloride).

Other useful cationic antimicrobial actives herein include biguamide compounds, either alone or in combination with other cationic antimicrobial actives. Suitable biguamide compounds include 1,1′-hexamethylene bis(5-(p-chlorophenyl)biguamide), commonly known as chlorhexidine, and its salts, e.g., with hydrochloric, acetic and gluconic acids. Other useful biguamide compounds include Cosmoci® CQ®, Vantocil®IB, including poly(hexamethylene biguamide) hydrochloride. Other useful cationic antimicrobial actives include the bis-biguamide alkanes. Usable water soluble salts of the above are chlorides, bromides, sulfates, alkyl sulfonates such as methyl sulfonate and ethyl sulfonate, phenylsulfonates such as p-methylphenyl sulfonates, nitrates, acetates, gluconates, and the like.

Examples of suitable bis-biguamide compounds are chlorhexidine; 1,6-bis-(2-ethylhexylbiguamidohexane)dihydrochloride; 1,6-di-(N1, N1′-phenyldiguamido-N5, N5′)-hexane tetrahydrochloride; 1,6-di-(N1, N1′-phenyl-N1, N1′-methyldiguamido-N5,N5′)-hexane dihydrochloride; 1,6-di(N1, N1′-o-chlorophenyldiguamido-N5, N5′)-hexane dihydrochloride; 1,6-di(N1, N1′-2,6-dichlorophenyldiguamido-N5, N5′)hexane dihydrochloride; 1,6-di[N1, N1′-β-(p-methoxyphenyl)diguamido-N5, N5′]-hexane dihydrochloride; 1,6-di(N1, N1′-α-methyl-β-phenyldiguamido-N5, N5′)-hexane dihydrochloride; 1,6-di(N1, N1′-p-nitrophenyldiguamido-N5, N5′)hexane dihydrochloride; ω:ω′-di-(N1, N1′-phenyldiguamido-N5, N5′)-di-n-propylether dihydrochloride; omega:omega′-di(N1,N1′-p-chlorophenyldiguamido-N5, N5′)-di-n-propylether tetrahydrochloride; 1,6-di(N1, N1′-2,4-dichlorophenyldiguamido-N5, N5′)hexane tetrahydrochloride; 1,6-di(N1, N1′-p-methylphenyldiguamido-N5, N5′)hexane dihydrochloride; 1,6-di(N1, N1′-2,4,5-trichlorophenyldiguamido-N5, N5′)hexane tetrahydrochloride; 1,6-di[N1, N1′α-(p-chlorophenyl) ethyldiguamido-N5, N5′]hexane dihydrochloride; ω:ω′di(N1, N1′-p-chlorophenyldiguamido-N5, N5′)m-xylene dihydrochloride; 1,12di(N1, N1′-p-chlorophenyldiguamido-N5, N5′)dodecane dihydrochloride; 1,10-di(N1, N1′-phenyldiguamido-N5, N5′)-decane tetrahydrochloride; 1,12-di(N1, N1′-phenyldiguamido-N5, N5′) dodecane tetrahydrochloride; 1,6-di(N1, N1′-o-chlorophenyldiguamido-N5, N5′) hexane dihydrochloride; 1,6-di(N1, N1′-p-chlorophenyldiguamido-N5, N5′)-hexane tetrahydrochloride; ethylene bis(1-tolyl biguamide); ethylene bis(p-tolyl biguamide); ethylene bis(3,5-dimethylphenyl biguamide); ethylene bis(p-tert-amylphenyl biguamide); ethylene bis(nonylphenyl biguamide); ethylene bis(phenyl biguamide); ethylene bis(N-butylphenyl biguamide); ethylene bis(2,5-diethoxyphenyl biguamide); ethylene bis(2,4-dimethylphenyl biguamide); ethylene bis(o-diphenylbiguamide); ethylene bis(mixed amyl naphthyl biguamide); N-butyl ethylene bis(phenylbiguamide); trimethylene bis(o-tolyl biguamide); N-butyl trimethylene bis(phenyl biguamide); and the corresponding pharmaceutically acceptable salts of all of the above such as the acetates; gluconates; hydrochlorides; hydrobromides; citrates; bisulfites; fluorides; polymaleates; N-coconutalkylsarcosinates; phosphites; hypophosphites; perfluorooctanoates; silicates; sorbates; salicylates; maleates; tartrates; fumarates; ethylenediaminetetraacetates; iminodiacetates; cinnamates; thiocyanates; arginates; pyromellitates; tetracarboxybutyrates; benzoates; glutarates; monofluorophosphates; and perfluoropropionates, and mixtures thereof.

Other useful antimicrobial agents include phenolic antibacterial agents, such as 2-hydroxydiphenyl compounds such as triclosan, available commercially under the tradename IRGASAN DP100®, from Ciba Specialty Chemicals Corp., Greensboro, N.C. Another useful 2-hydroxydiphenyl compound is 2,2′-dihydroxy-5,5′-dibromodiphenyl ether. Additional bisphenolic compounds are disclosed in U.S. Pat. No. 6,113,933, incorporated herein by reference. Other phenolic antimicrobials include, but are not limited to, chlorophenols (o-, m-, p-), 2,4-dichlorophenol, p-nitrophenol, picric acid, xylenol, p-chloro-m-xylenol, cresols (o-, m-, p-), p-chloro-m cresol, pyrocatechol, resorcinol, 4-n-hexylresorcinol, pyrogallol, phloroglucin, carvacrol, thymol, p-chlorothymol, o-phenylphenol, o benzylphenol, p-chloro-o-benzylphenol, phenol, 4-ethylphenol, and 4-phenolsulfonic acid. Other phenol derivatives are listed in WO 98/55096 and U.S. Pat. No. 6,113,933, incorporated herein by reference.

Suitable antimicrobial metal salts include salts of metals in groups 3b-7b, 8 and 3a-5a. Specifically are the salts of aluminum, zirconium, zinc, silver, gold, copper, lanthanum, tin, mercury, bismuth, selenium, strontium, scandium, yttrium, cerium, praseodymiun, neodymium, promethum, samarium, europium, gadolinium, terbium, dysprosium, holmium, erbium, thulium, ytterbium, lutetium and mixtures thereof. Suitable metallic antimicrobials include silver compounds as described in U.S. Pat. No. 6,180,584 to Sawan.

Suitable essential oil antimicrobials include those essential oils, which exhibit antimicrobial activity. By “actives of essential oils”, it is meant herein any ingredient of essential oils that exhibit anti-microbial activity. It is speculated that said anti-microbial essential oils and actives thereof act as proteins denaturing agents. Such anti-microbial essential oils include, but are not limited to, those obtained from thyme, lemongrass, citrus, lemons, orange, anise, clove, aniseed, pine, cinnamon, geranium, roses, mint, lavender, citronella, eucalyptus, peppermint, camphor, ajowan, sandalwood, rosmarin, vervain, fleagrass, lemongrass, ratanhiae, cedar and mixtures thereof. Suitable anti-microbial essential oils to be used herein are thyme oil, clove oil, cinnamon oil, geranium oil, eucalyptus oil, peppermint oil, citronella oil, ajowan oil, mint oil or mixtures thereof. Actives of essential oils to be used herein include, but are not limited to, thymol (present for example in thyme, ajowan), eugenol (present for example in cinnamon and clove), menthol (present for example in mint), geraniol (present for example in geranium and rose, citronella), verbenone (present for example in vervain), eucalyptol and pinocarvone (present in eucalyptus), cedrol (present for example in cedar), anethol (present for example in anise), carvacrol, hinokitiol, berberine, ferulic acid, cinnamic acid, methyl salicylic acid, methyl salycilate, terpineol, limonene and mixtures thereof. Suitable actives of essential oils to be used herein are thymol, eugenol, verbenone, eucalyptol, terpineol, cinnamic acid, methyl salicylic acid, limonene, geraniol or mixtures thereof.

Suitable oxidant antimicrobials include hydrogen peroxide and other peroxides, sources of hydrogen peroxide and other peroxides, generators of hydroxyl radical, peracid bleaches and peracid bleach precursors, as described in U.S. Pat. No. 6,548,467 to Baker et al. and U.S. Pat. No. 6,627,590 to Sherry et al. Other suitable oxidant antimicrobials include hypohalous acids and salts. These may be provided by a variety of sources, including compositions that lead to the formation of positive halide ions and/or hypohalite ions; hypohalous acid, hypohalous acid salt, hypohalous acid generating species, hypohalous acid salt generating species; as well as compositions that are organic based sources of halides, such as chloroisocyanurates, haloamines, haloimines, haloimides and haloamides, or mixtures thereof. These compositions may also produce hypohalous acid or hypohalite species in situ.

Suitable acid antimicrobials include: citric acid, cresylic acid, dodecylbenzene sulfonic acid, phosphoric acid, salicylic acid, sorbic acid, sulfamic acid, acetic acid, benzoic acid, boric acid, capric acid, caproic acid, cyanuric acid, dihydroacetic acid, dimethylsulfamic acid, propionic acid, polyacrylic acid, 2-ethyl-hexanoic acid, formic acid, fumaric acid, 1-glutamic acid, isopropyl sulfamic acid, naphthenic acid, oxalic acid, phosphorus acid, valeric acid, benzene sulfonic acid, xylene sulfonic acid, as well as any acid listed as a registered pesticide active ingredient with the United States Environmental Protection Agency. Further useful acids include: sulfonic acids, maleic acid, acetic acid, adipic acid, lactic acid, butyric acid, gluconic acid, malic acid, tartaric acid, as well as glycolic acid. Desirably glycolic acid and citric acid are used as they are effective and in plentiful supply.

Suitable concentrations of these antimicrobial agents in the chemical compositions range from greater than 5%, or from greater than 10%, or from about 0.5 to about 80%, or from about 1% to about 70%, or from about 5% to about 50%, or from about 10% to about 50%, by weight of the usage composition.

### Surfactants

The cleaning composition may contain one or more surfactants selected from anionic, nonionic, cationic, ampholytic, amphoteric and zwitterionic surfactants and mixtures thereof. A typical listing of anionic, nonionic, ampholytic, and zwitterionic classes, and species of these surfactants, is given in U.S. Pat. No. 3,929,678 to Laughlin and Heuring. A list of suitable cationic surfactants is given in U.S. Pat. No. 4,259,217 to Murphy. Where present, ampholytic, amphotenic and zwitteronic surfactants are generally used in combination with one or more anionic and/or nonionic surfactants.

### Anionic Surfactants

The cleaning composition may comprise an anionic surfactant. Essentially any anionic surfactants useful for detersive purposes can be comprised in the cleaning composition. These can include salts (including, for example, sodium, potassium, ammonium, and substituted ammonium salts such as mono-, di- and tri-ethanolamine salts) of the anionic sulfate, sulfonate, carboxylate and sarcosinate surfactants. Anionic surfactants may comprise a sulfonate or a sulfate surfactant. Anionic surfactants may comprise an alkyl sulfate, a linear or branched alkyl benzene sulfonate, or an alkyldiphenyloxide disulfonate, as described herein.

Other anionic surfactants include the isethionates such as the acyl isethionates, N-acyl taurates, fatty acid amides of methyl tauride, alkyl succinates and sulfosuccinates, monoesters of sulfosuccinate (for instance, saturated and unsaturated C12-C18 monoesters) diesters of sulfosuccinate (for instance saturated and unsaturated C6-C14 diesters), N-acyl sarcosinates. Resin acids and hydrogenated resin acids are also suitable, such as rosin, hydrogenated rosin, and resin acids and hydrogenated resin acids present in or derived from tallow oil. Anionic sulfate surfactants suitable for use herein include the linear and branched primary and secondary alkyl sulfates, alkyl ethoxysulfates, fatty oleoyl glycerol sulfates, alkyl phenol ethylene oxide ether sulfates, the C5-C17acyl-N—(C1-C4 alkyl) and —N—(C1-C2 hydroxyalkyl) glucamine sulfates, and sulfates of alkylpolysacchamides such as the sulfates of alkylpolyglucoside (the nonionic nonsulfated compounds being described herein). Alkyl sulfate surfactants may be selected from the linear and branched primary C10-C18 alkyl sulfates, the C11-C15 branched chain alkyl sulfates, or the C12-C14 linear chain alkyl sulfates.

Alkyl ethoxysulfate surfactants may be selected from the group consisting of the C10-C18 alkyl sulfates, which have been ethoxylated with from 0.5 to 20 moles of ethylene oxide per molecule. The alkyl ethoxysulfate surfactant may be a C11-C18, or a C11-C15 alkyl sulfate which has been ethoxylated with from 0.5 to 7, or from 1 to 5, moles of ethylene oxide per molecule. One aspect of the invention employs mixtures of the alkyl sulfate and/or sulfonate and alkyl ethoxysulfate surfactants. Such mixtures have been disclosed in PCT Patent Application No. WO 93/18124.

Anionic sulfonate surfactants suitable for use herein include the salts of C5-C20 linear alkylbenzene sulfonates, alkyl ester sulfonates, C6-C22 primary or secondary alkane sulfonates, C6-C24 olefin sulfonates, sulfonated polycarboxylic acids, alkyl glycerol sulfonates, fatty acyl glycerol sulfonates, fatty oleyl glycerol sulfonates, and any mixtures thereof. Suitable anionic carboxylate surfactants include the alkyl ethoxy carboxylates, the alkyl polyethoxy polycarboxylate surfactants and the soaps (‘alkyl carboxyls’), especially certain secondary soaps as described herein. Suitable alkyl ethoxy carboxylates include those with the formula RO(CH₂CH₂0)ₓCH₂COO⁻M⁺ wherein R is a C6 to C18 alkyl group, x ranges from 0 to 10, and the ethoxylate distribution is such that, on a weight basis, the amount of material where x is 0 is less than 20% and M is a cation. Suitable alkyl polyethoxypolycarboxylate surfactants include those having the formula RO—(CHR¹—CHR²-0)—R³ wherein R is a C6 to C18 alkyl group, x is from 1 to 25, R¹ and R² are selected from the group consisting of hydrogen, methyl acid radical, succinic acid radical, hydroxysuccinic acid radical, and mixtures thereof, and R³ is selected from the group consisting of hydrogen, substituted or unsubstituted hydrocarbon having between 1 and 8 carbon atoms, and mixtures thereof.

Suitable soap surfactants include the secondary soap surfactants, which contain a carboxyl unit connected to a secondary carbon. Suitable secondary soap surfactants for use herein are water-soluble members selected from the group consisting of the water-soluble salts of 2-methyl-1-undecanoic acid, 2-ethyl-1-decanoic acid, 2-propyl-1-nonanoic acid, 2-butyl-1-octanoic acid and 2-pentyl-1-heptanoic acid. Certain soaps may also be included as suds suppressors.

Other suitable anionic surfactants are the alkali metal sarcosinates of formula R—CON(R¹) CH—)COOM, wherein R is a C5-C17 linear or branched alkyl or alkenyl group, R¹ is a C1-C4 alkyl group and M is an alkali metal ion. Examples are the myristyl and oleoyl methyl sarcosinates in the form of their sodium salts.

### Nonionic Surfactants

Essentially any alkoxylated nonionic surfactants are suitable herein, for instance, ethoxylated and propoxylated nonionic surfactants. Alkoxylated surfactants can be selected from the classes of the nonionic condensates of alkyl phenols, nonionic ethoxylated alcohols, nonionic ethoxylated/propoxylated fatty alcohols, nonionic ethoxylate/propoxylate condensates with propylene glycol, and the nonionic ethoxylate condensation products with propylene oxide ethylene diamine adducts.

The condensation products of aliphatic alcohols with from 1 to 25 moles of alkylene oxide, particularly ethylene oxide and/or propylene oxide, are suitable for use herein. The alkyl chain of the aliphatic alcohol can either be straight or branched, primary or secondary, and generally contains from 6 to 22 carbon atoms. Also suitable are the condensation products of alcohols having an alkyl group containing from 8 to 20 carbon atoms with from 2 to 10 moles of ethylene oxide per mole of alcohol.

Polyhydroxy fatty acid amides suitable for use herein are those having the structural formula R²CONR¹Z wherein: R¹ is H, C1-C4 hydrocarbyl, 2-hydroxyethyl, 2-hydroxypropyl, ethoxy, propoxy, or a mixture thereof, for instance, C1-C4 alkyl, or C1 or C2 alkyl; and R² is a C5-C31 hydrocarbyl, for instance, straight-chain C5-C19 alkyl or alkenyl, or straight-chain C9-C17 alkyl or alkenyl, or straight-chain C11-C17 alkyl or alkenyl, or mixture thereof-, and Z is a polyhydroxyhydrocarbyl having a linear hydrocarbyl chain with at least 3 hydroxyls directly connected to the chain, or an alkoxylated derivative (for example, ethoxylated or propoxylated) thereof. Z may be derived from a reducing sugar in a reductive amination reaction, for example, Z is a glycityl.

Suitable fatty acid amide surfactants include those having the formula: R¹CON(R²)₂ wherein R¹ is an alkyl group containing from 7 to 21, or from 9 to 17 carbon atoms and each R² is selected from the group consisting of hydrogen, C1-C4 alkyl, C1-C4 hydroxyalkyl, and —(C₂H₄O)ₓH, where x is in the range of from 1 to 3.

Suitable alkylpolysaccharides for use herein are disclosed in U.S. Pat. No. 4,565,647 to Llenado, having a hydrophobic group containing from 6 to 30 carbon atoms and a polysaccharide, e.g., a polyglycoside, hydrophilic group containing from 1.3 to 10 saccharide units. Alkylpolyglycosides may have the formula: R²O(CₙH₂ₙO)ₜ(glycosyl)ₓ wherein R² is selected from the group consisting of alkyl, alkylphenyl, hydroxyalkyl, hydroxyalkylphenyl, and mixtures thereof in which the alkyl groups contain from 10 to 18 carbon atoms; n is 2 or 3; t is from 0 to 10, and x is from 1.3 to 8. The glycosyl may be derived from glucose.

### Amphoteric Surfactants

Suitable amphoteric surfactants for use herein include the amine oxide surfactants and the alkyl amphocarboxylic acids. Suitable amine oxides include those compounds having the formula R³(OR⁴)_{X}NO(R⁵)₂ wherein R is selected from an alkyl, hydroxyalkyl, acylamidopropyl and alkylphenyl group, or mixtures thereof, containing from 8 to 26 carbon atoms; R⁴ is an alkylene or hydroxyalkylene group containing from 2 to 3 carbon atoms, or mixtures thereof, x is from 0 to 5, preferably from 0 to 3; and each R⁵ is an alkyl or hydroxyalkyl group containing from 1 to 3, or a polyethylene oxide group containing from 1 to 3 ethylene oxide groups. Suitable amine oxides are C10-C18 alkyl dimethylamine oxide, and C10-18 acylamido alkyl dimethylamine oxide. A suitable example of an alkyl amphodicarboxylic acid is Miranol™ C2M Conc. manufactured by Miranol, Inc., Dayton, N.J.

Zwitterionic surfactants can also be incorporated into the cleaning compositions. These surfactants can be broadly described as derivatives of secondary and tertiary amines, derivatives of heterocyclic secondary and tertiary amines, or derivatives of quaternary ammonium, quaternary phosphonium or tertiary sulfonium compounds. Betaine and sultaine surfactants are exemplary zwittenionic surfactants for use herein.

Suitable betaines are those compounds having the formula R(R¹)₂N⁺R²COO⁻ wherein R is a C6-C18 hydrocarbyl group, each R¹ is typically C1-C3 alkyl, and R² is a C1-C5 hydrocarbyl group. Suitable betaines are C12-18 dimethyl-ammonio hexanoate and the C10-18 acylamidopropane (or ethane) dimethyl (or diethyl) betaines. Complex betaine surfactants are also suitable for use herein.

### Cationic Surfactants

Suitable cationic surfactants to be used herein include the quaternary ammonium surfactants. The quaternary ammonium surfactant may be a mono C6-C16, or a C6-C10 N-alkyl or alkenyl ammonium surfactant wherein the remaining N positions are substituted by methyl, hydroxyethyl or hydroxypropyl groups. Suitable are also the mono-alkoxylated and bis-alkoxylated amine surfactants.

Another suitable group of cationic surfactants, which can be used in the cleaning compositions, are cationic ester surfactants. The cationic ester surfactant is a compound having surfactant properties comprising at least one ester (i.e. —COO—) linkage and at least one cationically charged group. Suitable cationic ester surfactants, including choline ester surfactants, have for example been disclosed in U.S. Pat. Nos. 4,228,042, 4,239,660 and 4,260,529. The ester linkage and cationically charged group may be separated from each other in the surfactant molecule by a spacer group consisting of a chain comprising at least three atoms (i.e. of three atoms chain length), or from three to eight atoms, or from three to five atoms, or three atoms. The atoms forming the spacer group chain are selected from the group consisting, of carbon, nitrogen and oxygen atoms and any mixtures thereof, with the proviso that any nitrogen or oxygen atom in said chain connects only with carbon atoms in the chain. Thus spacer groups having, for example, —O—O— (i.e. peroxide), —N—N—, and —N—O— linkages are excluded, whilst spacer groups having, for example —CH₂—O—, CH₂— and —CH₂—NH—CH₂— linkages are included. The spacer group chain may comprise only carbon atoms, or the chain is a hydrocarbyl chain.

The cleaning composition may comprise cationic mono-alkoxylated amine surfactants, for instance, of the general formula: R¹R²R³N⁺ApR⁴X⁻ wherein R¹ is an alkyl or alkenyl moiety containing from about 6 to about 18 carbon atoms, or from 6 to about 16 carbon atoms, or from about 6 to about 14 carbon atoms; R² and R³ are each independently alkyl groups containing from one to about three carbon atoms, for instance, methyl, for instance, both R² and R³ are methyl groups; R⁴ is selected from hydrogen, methyl and ethyl; X⁻ is an anion such as chloride, bromide, methylsulfate, sulfate, or the like, to provide electrical neutrality; A is a alkoxy group, especially a ethoxy, propoxy or butoxy group; and p is from 0 to about 30, or from 2 to about 15, or from 2 to about 8. The ApR⁴ group in the formula may have p=1 and is a hydroxyalkyl group, having no greater than 6 carbon atoms whereby the —OH group is separated from the quaternary ammonium nitrogen atom by no more than 3 carbon atoms. Suitable ApR⁴ groups are —CH₂CH₂—0H, —CH₂CH₂CH₂—0H, —CH₂CH(CH₃)—OH and —CH(CH₃)CH₂—OH. Suitable R¹ groups are linear alkyl groups, for instance, linear R¹ groups having from 8 to 14 carbon atoms.

Suitable cationic mono-alkoxylated amine surfactants for use herein are of the formula R¹(CH₃)(CH₃)N⁺(CH₂CH₂0)₂₋₅HX⁻ wherein R¹ is C10-C18 hydrocarbyl and mixtures thereof, especially C10-C14 alkyl, or C10 and C12 alkyl, and X is any convenient anion to provide charge balance, for instance, chloride or bromide.

As noted, compounds of the foregoing type include those wherein the ethoxy (CH₂CH₂O) units (EO) are replaced by butoxy, isopropoxy [CH(CH₃)CH₂O] and [CH₂CH(CH₃)O] units (i-Pr) or n-propoxy units (Pr), or mixtures of EO and/or Pr and/or i-Pr units.

The cationic bis-alkoxylated amine surfactant may have the general formula: R¹R²N⁺ApR³A′qR⁴X⁻ wherein R¹ is an alkyl or alkenyl moiety containing from about 8 to about 18 carbon atoms, or from 10 to about 16 carbon atoms, or from about 10 to about 14 carbon atoms; R² is an alkyl group containing from one to three carbon atoms, for instance, methyl; R³ and R⁴ can vary independently and are selected from hydrogen, methyl and ethyl, X⁻ is an anion such as chloride, bromide, methylsulfate, sulfate, or the like, sufficient to provide electrical neutrality. A and A′ can vary independently and are each selected from C₁-C₄ alkoxy, for instance, ethoxy, (i.e., —CH₂CH₂O—), propoxy, butoxy and mixtures thereof, p is from 1 to about 30, or from 1 to about 4 and q is from 1 to about 30, or from 1 to about 4, or both p and q are 1.

Suitable cationic bis-alkoxylated amine surfactants for use herein are of the formula R¹CH₃N⁺(CH₂CH₂OH)(CH₂CH₂OH)X⁻, wherein R¹ is C10-C18 hydrocarbyl and mixtures thereof, or C10, C12, C14 alkyl and mixtures thereof, X⁻ is any convenient anion to provide charge balance, for example, chloride. With reference to the general cationic bis-alkoxylated amine structure noted above, since in one example compound R¹ is derived from (coconut) C12-C14 alkyl fraction fatty acids, R² is methyl and ApR³ and A′qR⁴ are each monoethoxy.

Other cationic bis-alkoxylated amine surfactants useful herein include compounds of the formula: R¹R²N⁺—(CH₂CH₂O)ₚH—(CH₂CH₂O)_{q}H X⁻ wherein R¹ is C10-C18 hydrocarbyl, or C10-C14 alkyl, independently p is 1 to about 3 and q is 1 to about 3, R² is C1-C3 alkyl, for example, methyl, and X⁻ is an anion, for example, chloride or bromide. Other compounds of the foregoing type include those wherein the ethoxy (CH₂CH₂O) units (EO) are replaced by butoxy (Bu) isopropoxy [CH(CH₃)CH₂O] and [CH₂CH(CH₃)O] units (i-Pr) or n-propoxy units (Pr), or mixtures of EO and/or Pr and/or i-Pr units.

### Fluorosurfactants

The inventive compositions may include at least one fluorosurfactant selected from nonionic fluorosurfactants, cationic fluorosurfactants, and mixtures thereof which are soluble or dispersible in the aqueous compositions being taught herein, sometimes compositions which do not include further detersive surfactants, or further organic solvents, or both. Suitable nonionic fluorosurfactant compounds are found among the materials presently commercially marketed under the tradename Fluorad® (ex. 3M Corp.) Exemplary fluorosurfactants include those sold as Fluorad® FC-740, generally described to be fluorinated alkyl esters; Fluorad® FC-430, generally described to be fluorinated alkyl esters; Fluorad® FC-431, generally described to be fluorinated alkyl esters; and, Fluorad® FC-170-C, which is generally described as being fluorinated alkyl polyoxyethlene ethanols.

Suitable nonionic fluorosurfactant compounds include those which is believed to conform to the following formulation: CₙF₂ₙ₊₁SO₂N(C₂H₅)(CH₂CH₂O)ₓCH₃ wherein: n has a value of from 1-12, or from 4-12, or 8; x has a value of from 4-18, or from 4-10, or 7; which is described to be a nonionic fluorinated alkyl alkoxylate and which is sold as Fluorad® FC-171 (ex. 3M Corp., formerly Minnesota Mining and Manufacturing Co.).

Additionally suitable nonionic fluorosurfactant compounds are also found among the materials marketed under the tradename ZONYL® (DuPont Performance Chemicals). These include, for example, ZONYL® FSO and ZONYL® FSN. These compounds have the following formula: RfCH₂CH₂—O—(CH₂CH₂O)ₓH where Rf is F(CF₂CF₂)_{y}. For ZONYL® FSO, x is 0 to about 15 and y is 1 to about 7. For ZONYL® FSN, x is 0 to about 25 and y is 1 to about 9.

An example of a suitable cationic fluorosurfactant compound has the following structure: CₙF₂ₙ₊₁SO₂NHC₃H₆N⁺(CH₃)₃I⁻ where n˜8. This cationic fluorosurfactant is available under the tradename Fluorad® FC-135 from 3M. Another example of a suitable cationic fluorosurfactant is F₃—(CF₂)ₙ—(CH₂)ₘSCH₂CHOH—CH₂—N⁺R₁R₂R₃ Cl⁻ wherein: n is 5-9 and m is 2, and R₁, R₂ and R₃ are —CH₃. This cationic fluorosurfactant is available under the tradename ZONYL® FSD (available from DuPont, described as 2-hydroxy-3-((gamma-omega-perfluoro-C₆₋₂₀-alkyl)thio)-N,N,N-trimethyl-1-propyl ammonium chloride). Other cationic fluorosurfactants suitable for use in the present invention are also described in EP 866,115 to Leach and Niwata.

The surfactants may be present at a level of from about 0.5% to 80%, or 1% to 50%, or 5% to 20% by weight.

### Solvent

Suitable organic solvents include, but are not limited to, C₁₋₆ alkanols, C₁₆ diols, C₁₋₁₀ alkyl ethers of alkylene glycols, C₃₋₂₄ alkylene glycol ethers, polyalkylene glycols, short chain carboxylic acids, short chain esters, isoparafinic hydrocarbons, mineral spirits, alkylaromatics, terpenes, terpene derivatives, terpenoids, terpenoid derivatives, formaldehyde, and pyrrolidones. Alkanols include, but are not limited to, methanol, ethanol, n-propanol, isopropanol, butanol, pentanol, and hexanol, and isomers thereof. Diols include, but are not limited to, methylene, ethylene, propylene and butylene glycols. Alkylene glycol ethers include, but are not limited to, ethylene glycol monopropyl ether, ethylene glycol monobutyl ether, ethylene glycol monohexyl ether, diethylene glycol monopropyl ether, diethylene glycol monobutyl ether, diethylene glycol monohexyl ether, propylene glycol methyl ether, propylene glycol ethyl ether, propylene glycol n-propyl ether, propylene glycol monobutyl ether, propylene glycol t-butyl ether, di- or tri-polypropylene glycol methyl or ethyl or propyl or butyl ether, acetate and propionate esters of glycol ethers. Short chain carboxylic acids include, but are not limited to, acetic acid, glycolic acid, lactic acid and propionic acid. Short chain esters include, but are not limited to, glycol acetate, and cyclic or linear volatile methylsiloxanes. Water insoluble solvents such as isoparafinic hydrocarbons, mineral spirits, alkylaromatics, terpenoids, terpenoid derivatives, terpenes, and terpenes derivatives can be mixed with a water-soluble solvent when employed.

Examples of organic solvent having a vapor pressure less than 0.1 mm Hg (20° C.) include, but are not limited to, dipropylene glycol n-propyl ether, dipropylene glycol t-butyl ether, dipropylene glycol n-butyl ether, tripropylene glycol methyl ether, tripropylene glycol n-butyl ether, diethylene glycol propyl ether, diethylene glycol butyl ether, dipropylene glycol methyl ether acetate, diethylene glycol ethyl ether acetate, and diethylene glycol butyl ether acetate (all available from ARCO Chemical Company).

The solvents can be present at a level of from 0.001% to 10%, or from 1% to 5% by weight.

### Additional Adjuncts

The cleaning compositions optionally contain one or more of the following adjuncts: stain and soil repellants, lubricants, odor control agents, perfumes, fragrances and fragrance release agents, and bleaching agents. Other adjuncts include, but are not limited to, acids, electrolytes, dyes and/or colorants, solubilizing materials, stabilizers, thickeners, defoamers, hydrotropes, cloud point modifiers, preservatives, and other polymers. The solubilizing materials, when used, include, but are not limited to, hydrotropes (e.g. water soluble salts of low molecular weight organic acids such as the sodium and/or potassium salts of toluene, cumene, and xylene sulfonic acid). The acids, when used, include, but are not limited to, organic hydroxy acids, citric acids, keto acid, and the like. Electrolytes, when used, include, calcium, sodium and potassium chloride. Thickeners, when used, include, but are not limited to, polyacrylic acid, xanthan gum, calcium carbonate, aluminum oxide, alginates, guar gum, clays, methyl, ethyl, and/or propyl hydroxycelluloses. Defoamers, when used, include, but are not limited to, silicones, aminosilicones, silicone blends, and/or silicone/hydrocarbon blends. Bleaching agents, when used, include, but are not limited to, peracids, hypohalite sources, hydrogen peroxide, and/or sources of hydrogen peroxide.

Preservatives, when used, include, but are not limited to, mildewstat or bacteriostat, methyl, ethyl and propyl parabens, short chain organic acids (e.g. acetic, lactic and/or glycolic acids), bisguanidine compounds (e.g. Dantagard® and/or Glydant®) and/or short chain alcohols (e.g. ethanol and/or IPA). The mildewstat or bacteriostat includes, but is not limited to, mildewstats (including non-isothiazolone compounds) include Kathon GC®, a 5-chloro-2-methyl-4-isothiazolin-3-one, KATHON ICP®, a 2-methyl-4-isothiazolin-3-one, and a blend thereof, and KATHON 886®, a 5-chloro-2-methyl-4-isothiazolin-3-one, all available from Rohm and Haas Company; BRONOPOL®, a 2-bromo-2-nitropropane 1, 3 diol, from Boots Company Ltd., PROXEL CRL®, a propyl-p-hydroxybenzoate, from ICI PLC; NIPASOL M®, an o-phenyl-phenol, Na⁺ salt, from Nipa Laboratories Ltd., DOWICIDE A®, a 1,2-Benzoisothiazolin-3-one, from Dow Chemical Co., and IRGASAN DP 200®, a 2,4,4′-trichloro-2-hydroxydiphenylether, from Ciba-Geigy A.G.

pH Control Agents

The cleaning composition may include a builder or buffer, which increase the effectiveness of the surfactant. The builder or buffer can also function as a softener and/or a sequestering agent in the cleaning composition. A variety of builders or buffers can be used and they include, but are not limited to, phosphate-silicate compounds, zeolites, alkali metal, ammonium and substituted ammonium polyacetates, trialkali salts of nitrilotriacetic acid, carboxylates, polycarboxylates, carbonates, bicarbonates, polyphosphates, aminopolycarboxylates, polyhydroxysulfonates, and starch derivatives.

Builders or buffers can also include polyacetates and polycarboxylates. The polyacetate and polycarboxylate compounds include, but are not limited to, sodium, potassium, lithium, ammonium, and substituted ammonium salts of ethylenediamine tetraacetic acid, ethylenediamine triacetic acid, ethylenediamine tetrapropionic acid, diethylenetriamine pentaacetic acid, nitrilotriacetic acid, oxydisuccinic acid, iminodisuccinic acid, mellitic acid, polyacrylic acid or polymethacrylic acid and copolymers, benzene polycarboxylic acids, gluconic acid, sulfamic acid, oxalic acid, phosphoric acid, phosphonic acid, organic phosphonic acids, acetic acid, glycolic acid, and citric acid. These builders or buffers can also exist either partially or totally in the hydrogen ion form.

The builder agent can include sodium and/or potassium salts of EDTA and substituted ammonium salts. The substituted ammonium salts include, but are not limited to, ammonium salts of methylamine, dimethylamine, butylamine, butylenesdiamine, propylamine, triethylamine, trimethylamine, monoethanolamine, diethanolamine, triethanolamine, isopropanolamine, ethylenediamine tetraacetic acid and propanolamine.

Buffering agents, when used, include, but are not limited to, organic acids, mineral acids, alkali metal and alkaline earth salts of silicate, metasilicate, polysilicate, borate, hydroxide, carbonate, carbamate, phosphate, polyphosphate, pyrophosphates, triphosphates, tetraphosphates, ammonia, hydroxide, monoethanolamine, monopropanolamine, diethanolamine, dipropanolamine, triethanolamine, and 2-amino-2-methylpropanol. Suitable buffering agents for compositions of this invention are nitrogen-containing materials. Some examples are amino acids such as lysine or lower alcohol amines like mono-, di-, and tri-ethanolamine. Other suitable nitrogen-containing buffering agents are tri(hydroxymethyl) amino methane (TRIS), 2-amino-2-ethyl-1,3-propanediol, 2-amino-2-methyl-propanol, 2-amino-2-methyl-1,3-propanol, disodium glutamate, N-methyl diethanolamide, 2-dimethylamino-2-methylpropanol (DMAMP), 1,3-bis(methylamine)-cyclohexane, 1,3-diaminopropanol N,N′-tetra-methyl-1,3-diamino-2-propanol, N,N-bis(2-hydroxyethyl)glycine (bicine) and N-tris(hydroxymethyl)methyl glycine (tricine). Other suitable buffers include ammonium carbamate, citric acid, acetic acid. Mixtures of any of the above are also acceptable. Useful inorganic buffers/alkalinity sources include ammonia, the alkali metal carbonates and alkali metal phosphates, e.g., sodium carbonate, sodium polyphosphate. For additional buffers see WO 95/07971, which is incorporated herein by reference. Other suitable pH adjusting agents include sodium or potassium hydroxide.

When employed, the builder or buffer comprises from about 0.001% to about 50% of the cleaning composition.

### Effervescence

The cleaning composition may comprise materials that effervesce when combined with water. The materials may be within a water-soluble, water-insoluble, or water-dispersible pouch to slow the effervescent action or to protect the composition from premature hydration. The materials may comprise a polymeric agent to slow the effervescence. One component of the effervescent materials may be an acidic material. Suitable for this purpose are any acids present in dry solid form. Suitable for this purpose are C2-20 organic mono- and poly-carboxylic acids such as alpha- and beta-hydroxycarboxylic acids; C2-20 organophosphorus acids such as phytic acid; C2-20 organosulfur acids such as toluene sulfonic acid; and peroxides such as hydrogen peroxide or materials that generate hydrogen peroxide in solution. Typical hydroxycarboxylic acids include adipic, glutaric, succinic, tartaric, malic, maleic, lactic, salicylic and citric acids as well as acid forming lactones such as gluconolactone and gluccrolactone. A suitable acid is citric acid. Also suitable as acid material may be encapsulated acids. Typical encapsulating material may include water-soluble synthetic or natural polymers such as polyacrylates (e.g. encapsulating polyacrylic acid), cellulosic gums, polyurethane and polyoxyalkylene polymers. By the term “acid” is meant any substance which when dissolved in deionized water at 1% concentration will have a pH of less than 7. These acids may also have a pH of less than 6.5 or less than 5. These acids may be at 25° C. in solid form, i.e. having melting points greater than 25° C. Concentrations of the acid should range from about 0.5 to about 80%, or from about 10 to about 65%, or from about 20 to about 45% by weight of the total composition.

Another component of the effervescent materials may be a alkaline material. The alkaline material may a substance that can generate a gas such as carbon dioxide, nitrogen or oxygen, i.e. effervesce, when contacted with water and the acidic material. Suitable alkaline materials are anhydrous salts of carbonates and bicarbonates, alkaline peroxides (e.g. sodium perborate and sodium percarbonate) and azides (e.g. sodium azide). An example of the alkaline material is sodium or potassium bicarbonate. Amounts of the alkaline material may range from about 1 to about 80%, or from about 5 to about 49%, or from about 15 to about 40%, or from about 25 to about 35% by weight of the total composition.

When the cleaning composition comprises effervescent materials, then the composition may comprise no more than 5%, or no more than 3.5%, or no more than 1% water by weight of the total composition. Water of hydration is not considered to be water for purposes of this calculation. However, water of hydration may be preferred or eliminated. The combined amount of acidic and alkaline materials may be greater than 1.5%, or from about 40 to about 95%, or from about 60 to about 80% by weight of the total composition.

### Fragrance

Compositions of the present invention may comprise from about 0.1% to about 20%, or 2% to 20%, or 3% to 20%, or 5% to 20% by weight of the fragrance oil. As used herein the term “fragrance oil” relates to the mixture of perfume raw materials that are used to impart an overall pleasant odor profile to a composition. As used herein the term “perfume raw material” relates to any chemical compound which is odiferous when in an un-entrapped state, for example in the case of pro-perfumes, the perfume component is considered, for the purposes of this invention, to be a perfume raw material, and the pro-chemistry anchor is considered to be the entrapment material. In addition “perfume raw materials” are defined by materials with a ClogP value preferably greater than about 0.1, more preferably greater than about 0.5, even more preferably greater than about 1.0. As used herein the term “ClogP” means the logarithm to base 10 of the octanol/water partition coefficient. This can be readily calculated from a program called “CLOGP” which is available from Daylight Chemical Information Systems Inc., Irvine Calif., U.S.A. Octanol/water partition coefficients are described in more detail in U.S. Pat. No. 5,578,563.

### Water

When the composition is an aqueous composition, water can be, along with the solvent, a predominant ingredient. The water can be present at a level of less than 99.9%, or less than about 99%, or less than about 98%. Deionized water is preferred. Where the cleaning composition is concentrated, the water may be present in the composition at a concentration of less than about 85 wt. %.

### Method of Use

The cleaning substrates can be used for cleaning, disinfectancy, or sanitization on inanimate, household surfaces, including toilets, floors, counter tops, furniture, windows, walls, and automobiles. Other surfaces include stainless steel, chrome, and shower enclosures. The cleaning substrate can be packaged individually or together in canisters, tubs, etc. The cleaning substrate can be used as part of a cleaning implement attached to a tool or motorized tool, such as one having a handle. Examples of tools using a cleaning substrate include U.S. Pat. No. 6,611,986 to Seals, PCT App. WO00/71012 to Belt et al., U.S. Pat. App. 2002/0129835 to Pieroni and Foley, and PCT App. WO00/27271 to Policicchio et al.

### EXAMPLES

### Simulated Hand Transfer Method

The amount of skin exposure to the antimicrobial agent in the cleaning substrate can be estimated using the Simulated Hand Transfer Method. Place one Whatman Filter Paper (GF/A 50 mm) in a Petri dish. Using deionized water, dropwise wet surface of filter paper with 1.8 g of water. The surface of the filter paper should be covered but not fully saturated. Place the test pad with a second Petri dish and 3 lb. weight on the filter paper for 2 seconds. Then remove the test pad. Extract the filter paper with 20 ml of a 50% methanol/50% water solvent mixture in a 50 ml centrifuge tube for 5 min on a wrist action shaker. Analyze the solvent mixture for the amount of transferred antimicrobial agent by an appropriate method, for example two-phase titration or HPLC.

### Transfer Measurement

The compositions in Table I were impregnated on an airlaid, high loft polyester nonwowen substrate of approximately 1 inch thickness and having an aluminosilicate/latex binder on the surface to provide scrubbiness. A plastic fitment was attached to one side of the substrate. The substrates were impregnated with cleaning composition containing XX g of quaternary ammonium antimicrobial agent. The substrates were measured for quaternary ammonium transfer by the Simulated Hand Trasfer Method. Sample C is a deformable, semi-solid gel with elastic modulus about 1000 kPa. The other samples approximate Newtonian fluids. The results show that Sample A has greater quaternary ammonium transfer, which should lead to greater skin irritation.

TABLE I

| | A | B | C |
|---|---|---|---|
| Benzyl and ethylbenzyl | | 16.250 | 43.820 |
| alkyldimethyl ammonium chloride^{a} | | | |
| Didecyl dimethyl ammonium | 34.400 | | |
| chloride^{b} | | | |
| EP-PO copolymer^{c} | | 29.167 | 44.944 |
| Alkyl polyglycoside^{d} | 9.445 | | 5.618 |
| Blue Dye | 0.8700 | 0.401 | 1.124 |
| Ethanol | 6.880 | | |
| Water balance | 48.405 | 54.182 | 4.494 |
| Viscosity @ 1/s | 35 cP | 180 cP | 8,000,000 cP |
| | | | (extrapolated) |
| Quat Transfer | 53 mg | 35 mg | 5 mg |

^{a}Barquat 4250Z ® from Lonza.
^{b}Bardac 2250 ® from Lonza.
^{c}Pluronic 77 ® from BASF.
^{d}APG 325N ® from Cognis.

### Cleaning Compositions

Examples of suitable cleaning compositions are provided in Tables II and III. The cleaning compositions can be loaded on the cleaning substrate in an amount of from 0.1 to 10.0 g of actives of cleaning composition on the cleaning substrate. The pH of the cleaning compostion can be measured by adding 10 g of the composition to 100 g of water.

TABLE II

| | D | E | F | G |
|---|---|---|---|---|
| APG 325N ® | 9.6 | 12.5 | 12.5 | 16.2 |
| Alkylbenzyl and | 33.0 | 15.5 | 22.6 | 5.0 |
| dialkyl dimethyl | | | | |
| ammonium chloride^{a} | | | | |
| Tetrapotassium | | 3.5 | | |
| EDTA^{b} | | | | |
| Glycolic acid | | | | 6.4 |
| Monoethanolamine | | | 2.3 | |
| Blue Dye | 0.9 | 0.2 | 0.1 | 0.5 |
| Fragrance | 8.1 | 6.0 | 6.0 | 2.0 |
| Water | balance | balance | balance | balance |
| PH | 7.5 | | 11.0 | 4.0 |

^{a}Bardac 205M ® from Lonza.
^{b}Versene K4 ® from Dow Chemical.

TABLE III

| | H | I | J | K | L | M | N |
|---|---|---|---|---|---|---|---|
| Alcohol ethoxylate^{a} | 41.9 | 44.4 | 41.1 | 41.1 | 34.0 | 50.0 | 30.0 |
| Versene K4 ® | 2.7 | 3.8 | 3.6 | 3.6 | | 8.0 | |
| APG 325N ® | 3.0 | 0.6 | 1.1 | 1.1 | 1.5 | 4.0 | 2.0 |
| Alkyl dimethyl | 3.0 | 3.3 | | | 5.0 | 4.0 | |
| benzyl ammonium | | | | | | | |
| chloride^{b} | | | | | | | |
| Glycolic acid | | | 8.7 | 9.9 | | | |
| Water (balance) | | | | | | | |

^{a}Surfonic L24-22 ® from Huntsman
^{b}Barquat MB50 ® from Lonza.

### Antimicrobial Effectiveness

The antimicrobial effectiveness of cleaning compositions in cleaning substrates was measured in Table IV using a modification of the “Use/Dilution Method for Testing of Disinfectants—Modification of AOAC Official Methods of Analysis, 15^{th} ed., 1990.” The test solution was obtained by swirling the cleaning substrate attached to a cleaning tool (as described in U.S. Pat. App. 2005/0066465 to Minkler et al.) in 2800 ml (full bowl) or 830 ml (empty bowl) of hard water (100 ppm).

TABLE IV

| | | | Disinfectancy | Sanitization |
|---|---|---|---|---|
| Chemical composition | Loading | Dilution | (Staph.) | (Staph.) |
| 22.6% Bardac 2250 | 3.1 g | 2800 ml | Pass | |
| 12.5% APG 325 | | | | |
| 6% Fragrance | | | | |
| 0.1% Dye | | | | |
| 4.5% | | | | |
| Monoethanolamine | | | | |
| 22.6% Bardac 2250 | 3.1 g | 2800 ml | | Pass |
| 12.5% APG 325 | | | | |
| 6% Fragrance | | | | |
| 0.1% Dye | | | | |
| 33.8% Bardac 2250 | 0.5 g | 830 ml | Pass | |
| 5.48% APG 325 | | | | |
| 9.3% Fragrance | | | | |
| 0.8% Dye | | | | |
| 7.1% | | | | |
| Monoethanolamine | | | | |

Without departing from the spirit and scope of this invention, one of ordinary skill can make various changes and modifications to the invention to adapt it to various usages and conditions. As such, these changes and modifications are properly, equitably, and intended to be, within the full range of equivalence of the following claims.

We claim:

1. A method of promoting a first product comprising;
a. using a statement comparing or directing a user to compare the irritation of the first product to a second product; and
b. using a description relating to the irritation of the first product;
c. wherein the first product comprises a cleaning substrate comprising an antimicrobial agent;
d. wherein the potential incidental hand transfer of the antimirobial agent from the substrate by the Simulated Hand Transfer Method is less for the first product than for the second product; and
e. wherein the first product is used to clean hard surfaces.

2. The method of claim 1; wherein the method additionally has the step of using directions to attach the substrate to a cleaning implement.

3. A method of reducing the skin irritation of a disposable substrate for use on a toilet cleaning tool comprising:
a. impregnating a cleaning composition in the substrate;
b. wherein the cleaning composition comprises greater than 5% antimicrobial agent;
c. wherein the cleaning composition has a viscosity greater than 100 cP; and
d. wherein the potential incidental hand transfer by the Simulated Hand Transfer Method is less than 50 mg of the antimicrobial agent.

4. A method of reducing the skin irritation of a disposable substrate for use on a cleaning implement comprising:
a. limiting the potential incidental hand transfer of an antimicrobial agent from the substrate by the Simulated Hand Transfer Method to less than 50 mg of the antimicrobial agent;
b. wherein the substrate is impregnated with a cleaning composition;
c. wherein the cleaning composition comprises the antimicrobial agent; and
d. wherein the cleaning composition has a viscosity greater than 100 cP.

5. The method of claim 4; wherein the viscosity is greater than 500 cP.

6. The method of claim 4; wherein the viscosity is greater than 1000 cP.

7. The method of claim 4; wherein the cleaning composition comprises greater than 5% antimicrobial agent.

8. The method of claim 4; wherein the cleaning composition comprises greater than 10% antimicrobial agent.

9. The method of claim 4; wherein the potential incidental hand transfer by the Simulated Hand Transfer Method is less than 40 mg of the antimicrobial agent.

10. The method of claim 4; wherein the potential incidental hand transfer by the Simulated Hand Transfer Method is less than 30 mg of the antimicrobial agent.

11. The method of claim 4; wherein the antimicrobial agent comprises an oxidant.

12. The method of claim 4; wherein the antimicrobial agent comprises a cationic antimicrobial compound.

13. The method of claim 4; wherein the antimicrobial agent comprises a quaternary ammonium compound.

14. The method of claim 4; wherein the antimicrobial agent comprises an essential oil.

15. The method of claim 4; wherein the antimicrobial agent comprises a phenolic.

16. The method of claim 4; wherein the antimicrobial agent comprises an antimicrobial metal salt.

17. The method of claim 4; wherein the substrate comprises a nonwoven.

18. The method of claim 4; wherein the substrate comprises a sponge.

19. The method of claim 4; wherein the cleaning composition comprises less than 85% water.
